(19) United States
(12) Patent Application Publication
(10) Pub. No.: US 2016/0022815 A1
(43) Pub. Date: Jan. 28, 2016

(54) Cures for Asthma, Psoriasis, Eczema, Epilepsy, Seizures, Soft Teeth, and Any Other Disease or Ailment Caused by Ascaris Parasites, Whether in Humans or Animals

(71) Applicant: Steven Allen Swan, Dixfield, ME (US)

(72) Inventor: Steven Allen Swan, Dixfield, ME (US)

(21) Appl. No.: 14/340,176

(22) Filed: Jul. 24, 2014

(51) Int. Cl. A61K 41/00

(52) U.S. Cl. CPC A61K 41/0004

(57) ABSTRACT

A new method of exterminating or repelling certain disease- or malady-causing parasites in humans and animals using the radio frequencies of human immune system defense chemicals benzoquinone and/or rhodizonic acid superimposed upon small bottles of plain water which is then ingested by the human or animal. The radio frequencies of human immune system defense chemicals superimposed upon small bottles of plain water create facsimiles of those human immune system defense chemicals.

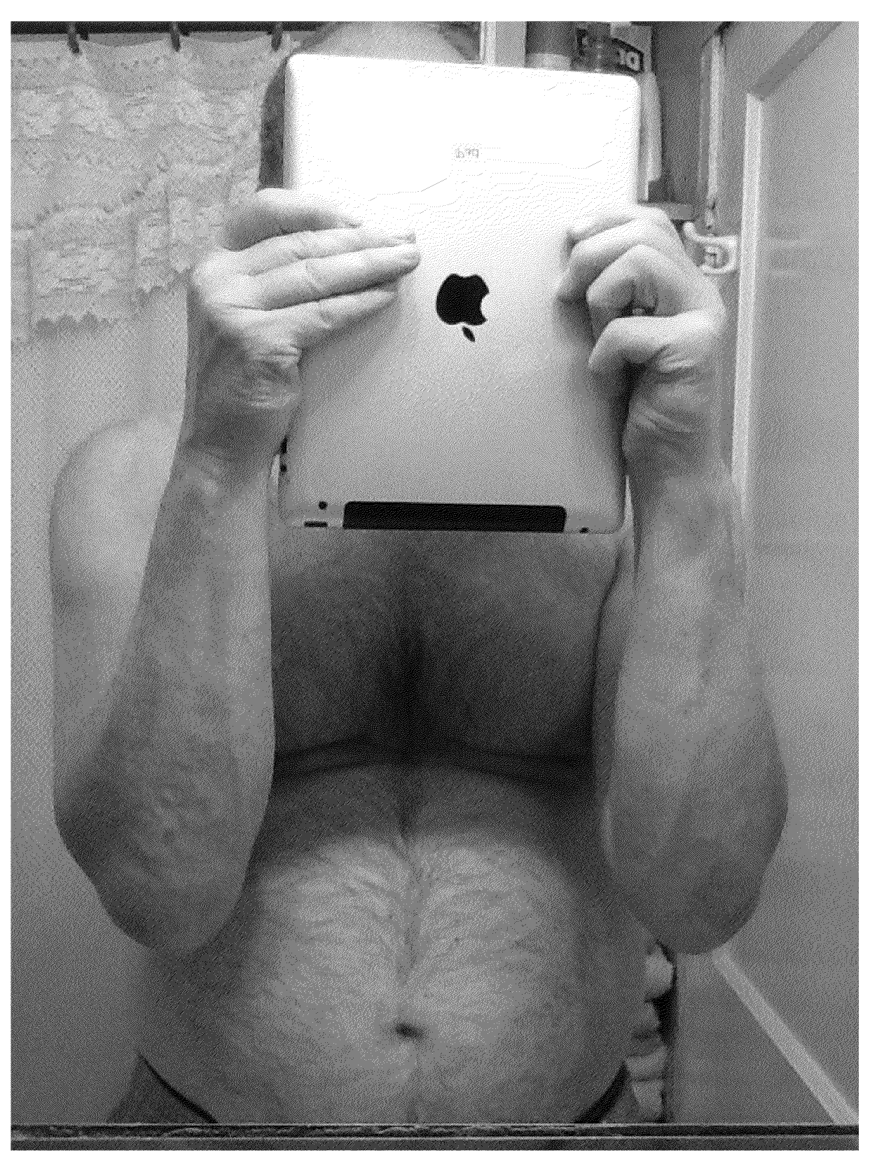

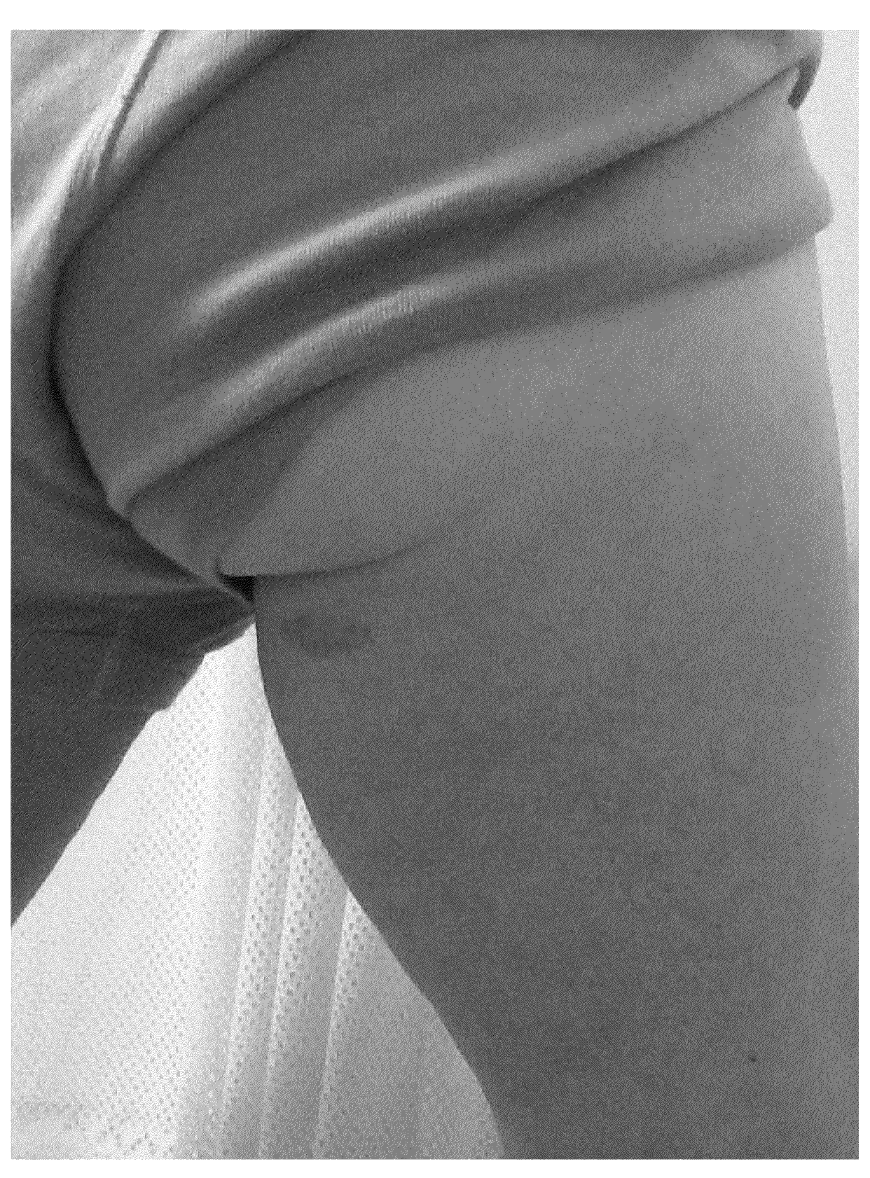

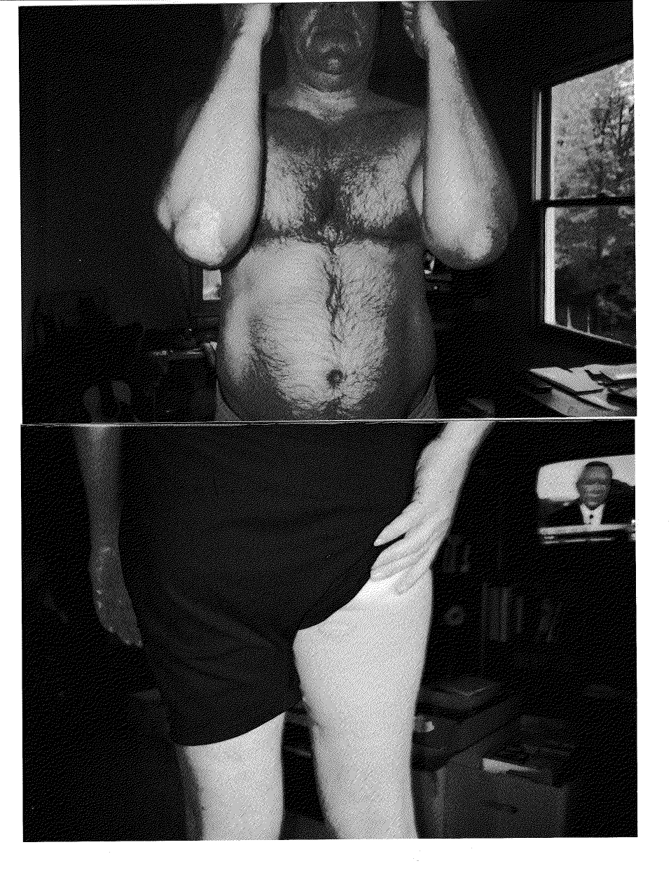

### CROSS-REFERENCE TO RELATED APPLICATIONS

Not Applicable

### STATEMENT REGARDING FEDERALLY-SPONSERED RESEARCH OR DEVELOPMENT (IF APPLICABLE)

Not Applicable

### REFERENCE TO SEQUENCE LISTING, A TABLE, OR A COMPUTER PROGRAM LISTING COMPACT DISK APPENDIX (IF APPLICABLE)

Not Applicable

### BACKGROUND OF THE INVENTION

The disease/ailment cures for which I am seeking patent protection consist of water upon which a radio frequency, a number of radio frequencies, or a range of radio frequencies have been superimposed. This process is based upon the discoveries of the late scientist Hulda Regehr Clark, Ph.D., N.D.

The cures being offered are true cures because they remove the primary causes of the disease or ailment by either exterminating or expelling them.

Dr. Clark discovered long ago that virtually everything in existence emits low-energy, high-frequency radio waves which can be used to identify that substance. (See The Cure For All Diseases, Hulda Regehr Clark, Ph.D., New Century Press, 1995, p. 5, as well as other sections of her books.) She also discovered that radio frequencies can be superimposed upon water and that the water will retain those frequencies for extended periods of time. (See The Cure For HIV and AIDS, Hulda Regehr Clark, New Century Press, 2003, p. 85, as well as other sections of her books.)

Dr. Clark discovered that the human disease/ailments of asthma, psoriasis, eczema, epilepsy, seizures, and soft teeth are all caused by chemicals produced by Ascaris parasites or their developmental stages that have invaded a patient's body. Most often, these diseases or ailments are caused by either Ascaris lumbricoides or Ascaris megalocephala, although there are other types of Ascaris, as well. When those parasites are eliminated from the body, the disease or ailment simply disappears as the body heals itself.

The substances used by the immune systems of humans to protect themselves from foreign invaders consist primarily of different types of white blood cells. However, the immune systems of humans and animals alike also utilize certain chemicals to protect themselves. One of those chemicals is called benzoquinone. Another is called rhodizonic acid. The chemicals produced by Ascaris overwhelm the organs affected and prevent them from producing their own defense chemicals.

Dr. Clark discovered that Ascaris lumbricoides and its developmental stages can be exterminated or repelled by benzoquinone and that Ascaris megalocephala and its stages can be exterminated or repelled by rhodizonic acid. She warned, however, that the actual chemicals benzoquinone and rhodizonic acid should never be ingested by a patient because they are too powerful and they are only produced by the body in picogram amounts. She also determined that the facsimile versions of these chemicals are perfectly safe.

Dr. Clark never published any information which specifically stated that the diseases or ailments asthma, psoriasis, eczema, epilepsy, seizures, soft teeth, or any other disease or ailment caused by Ascaris lumbricoides or Ascaris megalocephala infestations could be cured by ingesting plain water infused with the radio frequencies of the immune system defense chemicals benzoquinone and/or rhodizonic acid targeted to specific areas of a human or animal body.

When an organ is infested with Ascaris parasites or their developmental stages, the powerful chemicals produced by those parasites can overwhelm the chemicals produced by the immune system. Dr. Clark discovered that when the radio frequencies of the immune system chemicals are superimposed upon plain water and that plain water is ingested by a patient, those facsimile immune system chemicals counteract the chemicals produced by the parasites and nullify them.

Dr. Clark also discovered that the facsimile chemicals encourage the affected organ to renew the production of its own immune system chemicals. These actions result in the foreign invaders being exterminated and the affected organ returning back to its original healthy state, notwithstanding any other problems that might be present.

In addition to the ability of radio frequencies of foreign invaders to be superimposed upon plain water, the radio frequency range of organs can also be superimposed upon plain water. This ability allows the facsimile chemicals to be targeted to the specific organ affected.

### BRIEF SUMMARY OF THE INVENTION

Cures for asthma, psoriasis, eczema, epilepsy, seizures, soft teeth, and any other disease or ailment caused by Ascaris parasites using the radio frequencies of immune system defense chemicals superimposed upon plain water to create facsimiles of those immune system defense chemicals. These facsimiles are ingested by the affected human or animal.

### BRIEF DESCRIPTION OF THE SEVERAL VIEWS OF THE DRAWING

Not Applicable

### DETAILED DESCRIPTION OF THE INVENTION

### The Process for Exterminating Ascaris Parasites and Their Developmental Stages and Curing the Aforementioned Diseases and Ailments Asthma

In order to cure diseases or ailments caused by the infestation of Ascaris parasites, first one must determine the organ or organs within which the parasites are located. For asthma, this is simple—they are located in the lungs. For other diseases or ailments, it is not so simple to determine the location or locations. However, they can be determined by someone proficient in using a testing device invented by the late scientist Hulda Regehr Clark, Ph.D., N.D.

There are two (2) methods that can be employed in curing asthma:

1) A patient can forego determining which type of Ascaris parasite is causing his or her ailment and simply ingest both facsimile benzoquinone and facsimile rhodizonic acid. Using this method, the offending parasites will be exterminated or expelled.

2) A patient can hire someone proficient in using Dr. Clark's radio frequency analyzing apparatus to analyze a sample of the patient's saliva and determine which type of parasite is causing the illness. Then the patient can ingest the corresponding facsimile chemical, either benzoquinone or rhodizonic acid.

Once the person determines which method he or she wishes to utilize, he or she can purchase benzoquinone and/or rhodizonic acid targeted specifically to the lungs. To be more specific, each chemical will be targeted specifically to the right lung and to the left lung. However, one must ensure that the bottles of water infused with radio frequencies are never placed near magnets because their magnetic fields can destroy those infused frequencies. Nor should the bottles be refrigerated or frozen because doing so can jumble the frequencies and render them ineffective.

In summary, a person wishing to utilize the first method would purchase the following:

Benzoquinone for Right Lung

Benzoquinone for Left Lung

Rhodizonic Acid for Right Lung

Rhodizonic Acid for Left Lung

A person wishing to utilize the second method would purchase either benzoquinone or rhodizonic acid for right and left lungs, depending upon which type of Ascaris parasite is discovered.

The bottles of the facsimile chemicals come in ½ ounce dropper bottles. They are ingested by the drop as follows:
The patient is to have ingested no food or drink within either five (5) minutes before or five (5) minutes after taking the drops.
The patient tilts his head back and drops six (6) drops of the facsimile chemical under his tongue, just behind his lower front teeth. The patient is not to touch the bottle with his mouth, lest he risk contaminating it with germs.
The patient holds the drops in his mouth for one (1) minute before swallowing them, allowing them to mix with his or her saliva. After swallowing them, the person is to wait at least one (1) minute before ingesting the next set of drops.

The schedule for taking the drops is as follows:
Take them six (6) times per day for the first two (2) days.
After that, take them three (3) times per day until the bottle is empty.
Do not take any drops after sunset.

It should not take more than two (2) bottles of the facsimile chemicals, if that, before all of the Ascaris parasites in the affected organ are exterminated and the organ returns to being healthy again.

Among the many individuals and companies that have the ability to produce the facsimile chemicals is one in southern California called CTS Originals. Their website is www.ctsoriginals.com. They can also perform any radio frequency testing that might be required.

I used the foregoing method to cure my own asthma in June of 2011. It only took a few days and the asthma from which I had suffered for approximately 57 years completely disappeared! I used both benzoquinone and rhodizonic acid because I did not determine beforehand which type of Ascaris parasite was causing my asthma.

### Psoriasis and Eczema

In order to cure psoriasis or eczema, one must have some testing performed in order to determine which organ in the body the parasites are located. They could be anywhere.

The reason that the malady manifests in the skin is because the chemicals produced by the Ascaris parasites are very powerful and one function of the skin it to attempt to nullify any chemicals that might cause harm to any of our more vital organs like the brain, heart, liver, kidneys, etc. So the skin sequesters the chemicals and prevents them from causing other serious damage. The effect manifests itself as psoriasis or eczema in the skin.

I used this method to cure my own psoriasis in June of 2011. The testing determined that The Ascaris parasites that were causing my psoriasis were located in my spinal column (I did not ask the tester to determine which type of Ascaris was causing my psoriasis.) So I purchased facsimiles of both benzoquinone and rhodizonic acid targeted to my spinal column and used them as described above. Within a few days, the psoriasis from which I had suffered for approximately 37 years disappeared!

Testing can be accomplished by sending a saliva sample to the desired tester. They will provide you with their specific instructions for providing the sample.

I have first-hand proof that the above method is effective in curing psoriasis. It should also be effective in curing eczema since, according to Dr. Clark, Ascaris parasites are responsible for causing both diseases.

### Epilepsy and Seizures

With respect to epilepsy and seizures, the Ascaris parasites are located in the brain. So one would require either facsimile benzoquinone targeted to the brain or facsimile rhodizonic acid targeted to the brain, depending upon the testing results. Or the person could ingest both types of facsimile chemicals.

To be sure to get all of the Ascaris parasites, one might also wish to have the cerebrospinal fluid and the spinal cord tested because of their close proximity and relationship to the brain.

I do not have first-hand knowledge that this method will be effective in curing epilepsy and seizures as I do with asthma and psoriasis. However, since (according to Dr. Clark) Ascaris parasites are responsible for causing epilepsy and seizures, the foregoing method should be effective in curing these diseases/ailments also.

### Soft Teeth

According to Dr. Clark, the chemicals produced by Ascaris are powerful enough to cause a person's teeth to soften. An infected individual would want to have his saliva tested in order to determine the location of the Ascaris (and which type, if so desired) and then purchase and ingest the corresponding facsimile chemical targeted to that specific location.

I do not have first-hand knowledge that this method will be effective in curing soft teeth like I do with asthma and psoriasis. However, since (according to Dr. Clark) the chemicals produced by Ascaris parasites are responsible for causing teeth to soften, the foregoing method should be effective in curing this ailment also.

Other Diseases Caused by Ascaris Parasites (If Any), Including Simple Infestations

The foregoing method should be effective in curing any other diseases or ailments caused by Ascaris parasites, if any, including simple Ascaris infestations anywhere in the bodies of both humans and animals.

### Other Parasite-Killing Methods Invented by Dr. Clark

If there is anyone who is unable to attain the success that I achieved in curing asthma and psoriasis, they might try augmenting their efforts with other methods invented by Dr. Clark for exterminating or expelling Ascaris parasites. One of those methods is to deprive the Ascaris parasites of the nutrients they require in order to survive. The substance necessary for Ascaris lumbricoides to survive is called quercitin. To stop supplying quercitin, one must forego ingesting any undercooked squash or pumpkin products.

The substance necessary for Ascaris megalocephala to survive is called d-carnitine. To stop supplying d-carnitine, one must forego ingesting any commercially-raised meat, especially poultry. Organically-raised, free-range meat and poultry do not contain d-carnitine.

Another method is to use a device that she invented which produces square radio waves. She called her device a “Zapper” and determined that is most effective when the waves are produced at 30 KHz, although lower frequencies can also be effective.

The device is powered by a 9-volt battery and used with handholds connected to it. The patient can hold a handhold in each hand or wriststraps can be attached to each wrist. The radio waves travel throughout the body via the vascular and lymphatic systems. The radio waves exterminate a large number of parasites, bacteria, viruses, etc. The time required to do so is only seven (7) minutes.

However, this method only exterminates the foreign invaders that are not shielded from the radio waves, such as those in bowel contents, in testes, in ovaries, in eyeballs, in gallstones, etc. To reach those, Dr. Clark discovered that three (3) different types of herbs are effective.

In addition, Dr. Clark discovered that it takes more than one seven (7)-minute session in order to accomplish the objective of exterminating all parasites, bacteria, viruses, etc. This is because bacteria are smaller than and can fit inside parasites and viruses are smaller than and can fit inside bacteria. After the first seven (7) minute session, bacteria escape from the deceased parasite and can infect the patient. So another seven (7) minute session is required. Then viruses might escape from the deceased bacteria. So a third session is required. Therefore, in order for a zapping session to be effective, it should consist of the following:
One seven (7) minute zapping session
A twenty (20) minute rest period
A second seven (7) minute zapping session
Another twenty (20) minute rest period
A third seven (7) minute zapping session

A person can zap as many times per day as he or she wishes. However, each complete session should consist of all three (3) smaller sessions.

Another zapping method that can be used to augment the cure that I am offering is called “plate-zapping”. This method also utilizes a Clark-invented zapper. However, the radio waves are targeted to the affected organ only.

This is accomplished by introducing into the zapper circuit a bottle of facsimile water (described above) which contains the radio frequency of the affected organ superimposed upon it. The bottle is placed upon an aluminum plate that has been inserted into the positive (as opposed to negative) section of the circuit coming out of the zapper.

Plate-zapping is accomplished with only one (1) twenty (20) minute session. However, the 9-volt battery used must not go below 8.9 volts by the end of the twenty (20) minute session, otherwise the session will not be effective.

A person may plate-zap as many times per day as he or she wishes.

### Other Considerations

When parasites are exterminated while they are within a digestive organ, the remains will simply pass out of the body. However, when parasites are within organs other than digestive organs when they are exterminated, their remains begin to decompose while in that organ. To prevent any problems that might occur because of this (such as fungal infections), a person should take multiple digestive enzymes, as well as the digestive enzymes pancreatin and lipase.

In addition, Dr. Clark discovered that in order for our immune systems (our white blood cells) to function effectively in removing the remnants of the deceased parasites, bacteria, viruses, etc., they require three (3) nutrients—organic vitamin C, organic germanium, and organic selenium. A person using the method described here to cure his or her disease or ailment should also take adequate amounts of these nutrients.

1. A new method for curing asthma, psoriasis, eczema, epilepsy, seizures, soft teeth, and possibly other diseases or maladies in humans caused by Ascaris parasites by exterminating or repelling those parasites by ingesting water superimposed with the radio frequencies of human immune system defense chemicals benzoquinone or rhodizonic acid. Water superimposed with the radio frequencies of human immune system defense chemicals benzoquinone or rhodizonic acid is a facsimile of those defense chemicals. These facsimiles can also be used to eliminate Ascaris parasitism in animals.
